(19) United States
(12) Patent Application Publication
(10) Pub. No.: US 2007/0117827 A1
(43) Pub. Date: May 24, 2007

(54) Compositions for affecting weight loss

(76) Inventors: Gary Tollefson, Indianapolis, IN (US); Anthony McKinney, San Diego, CA (US); Michelle S. Higgin, Apex, NC (US); John M. McCall, Boca Grande, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/492,685

(22) Filed: Jul. 24, 2006

Related U.S. Application Data
(60) Provisional application No. 60/702,877, filed on Jul. 27, 2005.

(51) Int. Cl. A61K 31/485; A61K 31/165

(52) U.S. Cl. 514/282; 514/618

(57) ABSTRACT

Disclosed are compositions for affecting weight loss comprising a first compound and a second compound, where the first compound is a metabolite of naltrexone, such as 6-β-naltrexol or a prodrug of a naltrexone metabolite, and the second compound causes increased agonism of a melanocortin 3 receptor (MC3-R) and/or a melanocortin 4 receptor (MC4-R), and/or increases the concentration of α-MSH in the central nervous system. Also disclosed are methods of affecting weight loss, increasing energy expenditure, increasing satiety in an individual, or suppressing the appetite of an individual, comprising identifying an individual in need thereof and treating that individual to antagonize opioid receptor activity with a metabolite of naltrexone, such as 6-β-naltrexol or a prodrug of a naltrexone metabolite, and treating that individual to enhance α-MSH activity, e.g., by administration of a second compound causes increased agonism of MC3-R and/or MC4-R, and/or increases the concentration of α-MSH in the central nervous system.

### RELATED APPLICATION INFORMATION

This application claims priority to U.S. Provisional Patent Application Ser. No. 60/702,877, filed Jul. 27, 2005, which is hereby incorporated by reference in its entirety.

### BACKGROUND OF THE INVENTION

1. Field of the Invention

The present invention is in the field of pharmaceutical compositions and methods for the treatment of obesity and for affecting weight loss in individuals.

2. Description of the Related Art

Obesity is a disorder characterized by the accumulation of excess fat in the body. Obesity has been recognized as one of the leading causes of disease and is emerging as a global problem. Increased instances of complications such as hypertension, non-insulin dependent diabetes mellitus, arteriosclerosis, dyslipidemia, certain forms of cancer, sleep apnea, and osteoarthritis have been related to increased instances of obesity in the general population.

Obesity has been defined in terms of body mass index (BMI). BMI is calculated as weight (kg)/[height (m)]². According to the guidelines of the U.S. Centers for Disease Control and Prevention (CDC) and the World Health Organization (WHO), for adults over 20 years old, BMI falls into one of these categories: below 18.5 is considered underweight, 18.5-24.9 is considered normal, 25.0-29.9 is considered overweight, and 30.0 and above is considered obese (World Health Organization. Physical status: The use and interpretation of anthropometry. Geneva, Switzerland: World Health Organization 1995. WHO Technical Report Series).

Prior to 1994, obesity was generally considered a psychological problem. The discovery of the adipostatic hormone leptin in 1994 brought forth the realization that, in certain cases, obesity may have a biochemical basis (Zhang et al., “Positional cloning of the mouse obese gene and its human homologue,” Nature, 372:425-432 (1994)). A corollary to this realization was the idea that the treatment of obesity may be achieved by chemical approaches. Since then, a number of such chemical treatments have entered the market. The most famous of these attempts was the introduction of Fen-Phen, a combination of fenfluramine and phentermine. Unfortunately, it was discovered that fenfluramine caused heart-valve complications, which in some cases resulted in the death of the user. Fenfluramine has since been withdrawn from the market. There has been some limited success with other combination therapy approaches, particularly in the field of psychological eating disorders. One such example is Devlin, el al., Int. J. Eating Disord. 28:325-332 (2000), in which a combination of phentermine and fluoxetine showed some efficacy in the treatment of binge eating disorders.

In addition to those individuals who satisfy a strict definition of medical obesity, a significant portion of the adult population is overweight. These overweight individuals, as well as individuals at risk for becoming overweight or obese, would also benefit from the availability of an effective weight-loss composition. Therefore, there is an unmet need in the art to provide pharmaceutical compositions that can prevent weight gain and/or affect weight loss without having significant adverse side effects.

### SUMMARY OF THE INVENTION

An embodiment provides compositions for affecting weight loss, increasing energy expenditure, increasing satiety in an individual, and/or suppressing the appetite of an individual, comprising a first compound and a second compound, where the first compound is a metabolite of naltrexone and the second compound causes increased agonism of a melanocortin 3 receptor (MC3-R) and/or a melanocortin 4 receptor (MC4-R), and/or increases the concentration of α-MSH in the central nervous system. The metabolite of naltrexone may be naltrexol (e.g., 6-β-naltrexol) or a compound of the formula (II):

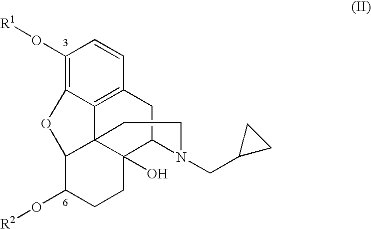

wherein at least one of R¹ and R² in formula (II) is a PO₃H group or a salt thereof, or an organic group containing from 2 to 20 carbons, preferably 3 to 20 carbons, that is selected to form a 3-O-ester derivative, a 6-O-ester derivative, a 3-O,6-O-diester derivative, a 3-carbamate derivative, a 6-carbamate derivative, a 3,6-dicarbamate derivative, a 3-carbonate derivative, a 6-carbonate derivative, or a 3,6-dicarbonate derivative. Preferably, one or neither of R¹ and R² is H. For example, in an embodiment, one or neither of R¹ and R2 is H and at least one of R¹ and R² is a group selected from the following:

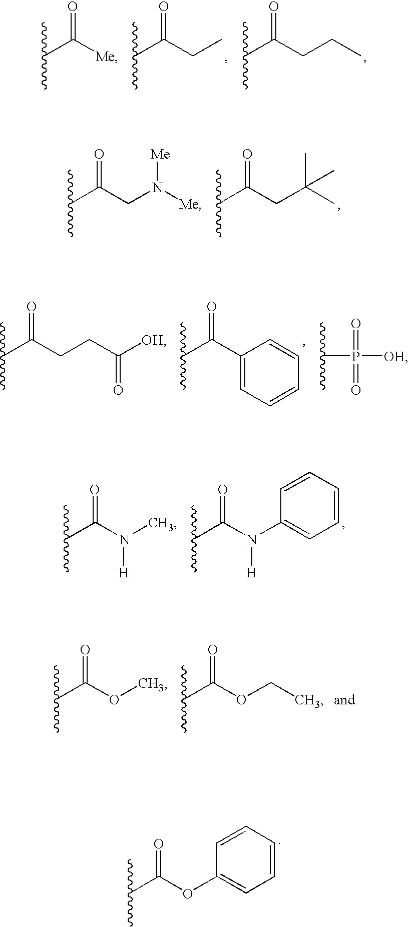

Another embodiment provides methods of affecting weight loss, increasing energy expenditure, increasing satiety in an individual, and/or suppressing the appetite of an individual, comprising identifying an individual in need thereof, treating that individual to antagonize opioid receptor activity with a metabolite of naltrexone, and treating the individual to enhance α-MSH activity. In an embodiment, treating the individual comprises administering a first compound that is a naltrexone metabolite and a second compound that increases agonism of MC3-R and/or MC4-R, and/or increases the concentration of α-MSH in the central nervous system. In some embodiments the first and second compounds are administered substantially simultaneously, e.g., by separate substantially simultaneous administration of the two compounds or by administration of the composition described above. In other embodiments, the two compounds are administered sequentially, in either order. The metabolite of naltrexone may be a naltrexol (e.g., 6-β-naltrexol) or a compound of the formula (II).

Another embodiment provides novel compounds of the formula (II), wherein one or neither of R¹ and R2 is H and wherein at least one of R¹ and R² is a group selected from the following:

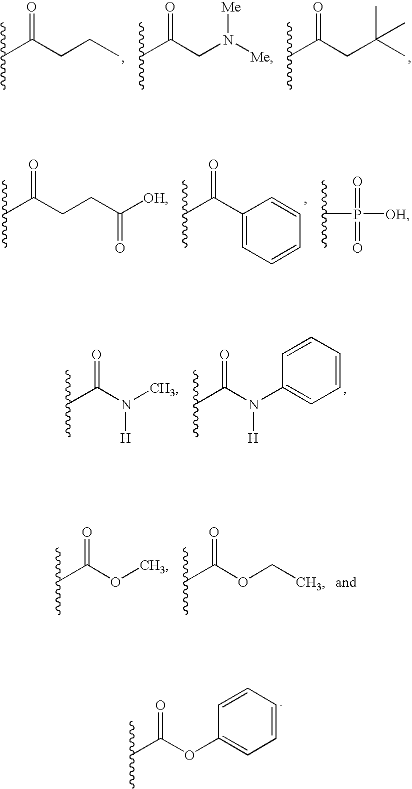

Another embodiment provides a composition for affecting weight loss, increasing energy expenditure, increasing satiety in an individual, and/or suppressing the appetite of an individual, comprising an effective amount of a compound of formula (II) and a pharmaceutically acceptable carrier. In preferred embodiments, the compound of formula (II) is a naltrexone metabolite (e.g., a prodrug of a naltrexone metabolite). In some embodiments, the compound of formula (II) is a novel compound as described above. In some embodiments, the composition further comprises a second compound that increases agonism of MC3-R and/or MC4-R, and/or increases the concentration of α-MSH in the central nervous system.

Another embodiment provides a method of affecting weight loss, increasing energy expenditure, increasing satiety in an individual, and/or suppressing the appetite of an individual, comprising identifying an individual in need thereof, and administering an effective amount of a compound of formula II, e.g., by administering a composition that comprises a compound of formula (II) and a pharmaceutically acceptable carrier as described above. In preferred embodiments, the method further comprises administering an effective amount of a second compound that increases agonism of MC3-R and/or MC4-R, and/or increases the concentration of α-MSH in the central nervous system, e.g., by administering a composition that comprises the compound of formula (II), the second compound, and the pharmaceutically acceptable carrier as described above.

In some embodiments described herein, the first compound comprises 6-β-naltrexol and/or a prodrug of a naltrexone metabolite (such as a compound of formula II). In some embodiments disclosed herein, the first compound can antagonize an opioid receptor in a mammal, where the opioid receptor is selected from a group consisting of a μ-opioid receptor, a κ-opioid receptor, and a δ-opioid receptor.

In some of the embodiments described herein, the second compound is at least one selected from a selective serotonin reuptake inhibitor (SSRI), a serotonin 2C agonist, and a serotonin 1B agonist. In some embodiments, the second compound is at least one selected from fluoxetine, fluvoxamine, sertraline, paroxetine, citalopram, escitalopram, sibutramine, duloxetine, and venlafaxine, and pharmaceutically acceptable salts or prodrugs thereof. In other embodiments the second compound is selected from sumatriptan, almotriptan, naratriptan, frovatriptan, rizatriptan, zomitriptan, and elitriptan. In some embodiments, the second compound is selected from a γ-amino butyric acid (GABA) inhibitor, a GABA receptor antagonist, a GABA channel antagonist and an anticonvulsant. In some embodiments, the second compound is selected from zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide. In some embodiments, the second compound is selected from a dopamine agonist, a dopamine reuptake inhibitor, a norepinephrine reuptake inhibitor, a norepinephrine releaser, and a norepinephrine agonist. In some embodiments, the second compound is selected from the group consisting of cabergoline, amantadine, lisuride, pergolide, ropinirole, pramipexole, bromocriptine, phentermine, bupropion, thionisoxetine, reboxetine, diethylpropion, phendimetrazine and benzphetamine. In any of the embodiments described herein, the second compound can be bupropion.

These and other embodiments are described in greater detail below.

### DETAILED DESCRIPTION OF THE PREFERRED EMBODIMENTS

Arcuate nucleus neurons are responsive to a wide array of hormones and nutrients, including leptin, insulin, gonadal steroids, and glucose. In addition to potential transport mechanisms, peripheral substances may access these neurons via arcuate cell bodies in and projections to the median eminence, a region considered to be a circumventricular organ, which lacks a blood-brain barrier (Cone et al., “The arcuate nucleus as a conduit for diverse signals relevant to energy homeostasis,” Int'l Journal of Obesity 25, Suppl 5, S63-S67 (2001)).

Administration of exogenous leptin activates a number of different neurons in hypothalamic and brainstem cell groups that bear leptin receptor. Leptin-responsive neurons in the arcuate nucleus include both those containing neuropeptide Y (NPY) and agouti-related peptide (AgRP) in the medial part of the nucleus and those containing both pro-opiomelanocortin (POMC) and its derivatives, including α-melanocyte stimulating hormone (α-MSH), as well as cocaine and amphetamine-related transcript (CART) (Saper et al., “The need to feed: Homeostatic and hedonic control of eating,” Neuron, 36:199-211 (2002)).

The leptin-responsive POMC neurons in the arcuate nucleus are believed to cause anorexia and weight reduction by the action of α-MSH on MC3-R and/or MC4-R. The highest MC3-R expression level is in the hypothalamus and limbic system, whereas MC4-R mRNA is expressed in virtually all major brain regions. Some of the metabolic effects resulting from stimulation of MC4-R are decreased food intake and an increase in energy expenditure through stimulation of thyrotropin-releasing hormone and activation of the sympathetic nervous system. Targeted deletion of the MC4-R gene produces obesity, hyperphagia, hyperinsulinemia, and reduced energy expenditure. Targeted deletion of MC3-R results in increased adiposity due to decreased energy expenditure (Korner et al., “The emerging science of body weight regulation and its impact on obesity treatment,” J. Clin. Invest. 111(5):565-570 (2003)). Thus, it is believed that increased concentrations of α-MSH in the central nervous system (CNS) increases its action on MC3-R and/or MC4-R, resulting in a suppressed appetite.

POMC neurons also release β-endorphin when they release α-MSH. β-endorphin is an endogenous agonist of the μ-opioid receptors (MOP-R), found on the POMC neurons. Stimulation of MOP-R decreases the release of α-MSH. This is a biofeedback mechanism that under normal physiological conditions controls the concentration of α-MSH in the CNS. Thus, blocking MOP-R by opioid antagonists will break the feedback mechanism, which results in continued secretion of α-MSH and an increase in its concentration in the CNS.

A second population of neurons in the arcuate nucleus tonically inhibits the POMC neurons. These POMC-inhibiting neurons secrete NPY, the neurotransmitter γ-aminobutyric acid (GABA), and AgRP. NPY and GABA inhibit POMC neurons, via NPY Y1 receptors and GABA receptors, respectively. Thus, within the arcuate nucleus NPY and GABA inhibit the release of α-MSH, and therefore are stimulators of feeding. It is known that leptin inhibits the release of GABA from NPY terminals synapsing onto POMC neurons, whereas ghrelin, an orexigenic peptide, stimulates the ghrelin receptors on NPY neurons and increase the secretion of NPY and GABA onto the POMC cells, which in turn inhibits the release of α-MSH.

AgRP stimulates food intake in the rat through antagonism of the interaction of α-MSH at MC4-R. Expression of the AgRP gene is suppressed by leptin.

Serotonin, also known as 5-hydroxytryptamine or 5-HT, activates the POMC neurons to secrete α-MSH. However, serotonin is taken up and removed from action by specific transporters so that a single serotonin molecule has short term effects. It is known that selective serotonin re-uptake inhibitors (SSRIs) prevent the uptake of serotonin and increase its concentrations in the CNS. Thus, SSRIs also increase the secretion of α-MSH and its concentrations in the CNS.

Dopamine also increases the activity of POMC neurons to secrete α-MSH. Like serotonin, dopamine is also taken up and removed from action so that a single dopamine molecule has short term effect. Dopamine re-uptake inhibitors, which prevent or reduce the uptake of dopamine, can also increase the secretion of α-MSH and its concentrations in the CNS.

Therefore, increased secretion of α-MSH through various mechanisms, such as serotonin re-uptake inhibition, are among the strategies that the methods and pharmaceutical compositions of the present invention can involve to produce a biochemical anorexigenic effect.

In some aspects, the present invention provides a multi-faceted combination therapy approach to the problem of weight loss. It addresses not just single molecules, messengers, or receptors, but instead acts on multiple points in the feeding and satiety pathway. Aspects of the present invention are directed to increasing the concentrations of α-MSH in the CNS by stimulating the release of α-MSH, suppressing its metabolism, reducing the antagonism of its interaction at MC3/4-R, and suppressing feedback mechanisms that slow or stop its release. Aspects of the present invention include pharmaceutical compositions whose components achieve one or more of these functions. The present inventors have discovered that a combination of two or more of the compounds disclosed herein results in a synergistic effect that affects weight loss more quickly and on a more permanent basis. Those skilled in the art will appreciate the various discussions of the mechanisms for appetite suppression and/or weight loss provided herein, and recognize that those disclosures do not restrict the scope of the inventions described herein, which are not limited by theory of operation.

Various combinations have been disclosed as being useful for helping in promoting weight loss. For example, a composition for the treatment of obesity can comprise an opioid antagonist and a compound that causes increased agonism of MC3-R or MC4-R compared to normal physiological conditions, see U.S. Patent Publication No. 2004-0254208, published Dec. 16, 2004, which is hereby incorporated by reference in its entirety.

The present inventors have realized that particular opioid antagonists, such as metabolites of naltrexone, for example, 6-β-naltrexol, can have especially beneficial properties when used in combination with an agent that enhances alpha-MSH activity.

Thus, an embodiment provides a composition for the treatment of obesity and/or for affecting weight loss, comprising a first compound and a second compound, where the first compound is a metabolite of naltrexone, or a pharmaceutically acceptable salt or prodrug thereof, and the second compound enhances alpha-MSH activity. Similarly, a method of promoting weight loss using the metabolite of naltrexone is also contemplated. In some embodiments, the metabolite of naltrexone is administered alone or without other weight loss promoting compounds in order to promote weight loss. In other embodiments, the metabolite of naltrexone is administered with an anticonvulsant, such as zonisamide or topiramate.

In certain embodiments, the metabolite of naltrexone is 6-β-naltrexol. The chemical structure of 6-β-naltrexol is shown below in Formula (I).

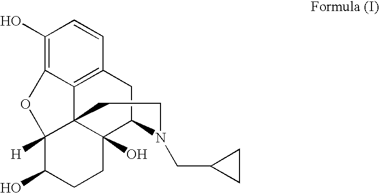

In certain embodiments, the second compound causes increased activity of the POMC neurons, leading to greater agonism at MC3-R and/or MC4-R.

In certain embodiments, the metabolite of naltrexone antagonizes a μ-opioid receptor (MOP-R) in a mammal. The mammal can be selected from the group consisting of mice, rats, rabbits, guinea pigs, dogs, cats, sheep, goats, cows, primates, such as monkeys, chimpanzees, and apes, and humans.

A method for synthesizing 6-β-naltrexol is described in U.S. Pat. No. 4,089,855. Additionally, a discussion of the use of 6-β-naltrexol in the treatment of drug dependency can be found in U.S. Pat. No. 6,713,488. Both U.S. Pat. Nos. 4,089,855 and 6,713,488 are hereby incorporated by reference herein in their entireties, including any drawings.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound with an acid such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound with a base to form a salt such as an ammonium salt, an alkali metal salt (such as a sodium or a potassium salt), an alkaline earth metal salt (such as a calcium or a magnesium salt), a salt of an organic base (such as dicyclohexylamine, N-methyl-D-glucamine, and/or tris(hydroxymethyl) methylamine salt), or a salt of an amino acid (such as an arginine or lysine salt).

A “prodrug” refers to an agent that is converted into the parent drug in vivo. Prodrugs are often useful because, in some situations, they may be easier to administer than the parent drug. They may, for instance, be more bioavailable by oral administration than the parent. The prodrug may also have improved solubility in pharmaceutical compositions over the parent drug, or may demonstrate increased palatability or be easier to formulate. An example, without limitation, of a prodrug is an ester of a naltrexone metabolite that facilitates transmittal across a cell membrane where water solubility is detrimental to mobility but which then is metabolically hydrolyzed to the carboxylic acid, the active entity, once inside the cell where water-solubility is beneficial. A further example of a prodrug is a naltrexone metabolite containing a short peptide (polyaminoacid) bonded to an acid group where the peptide is metabolized to provide the active moiety.

References herein to compositions containing metabolites of naltrexone or methods of using such naltrexone metabolites will be understood by those skilled in the art to include prodrugs of those metabolites unless the context clearly dictates otherwise. For example, in an embodiment, a naltrexone metabolite useful in the methods and/or compositions described herein is a naltrexol prodrug of the general formula (II):

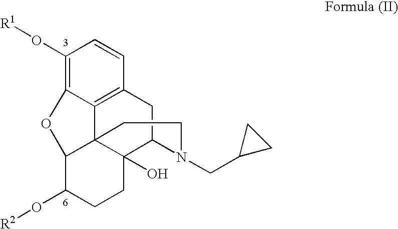

In formula (II), at least one of R¹ and R² is a PO₃H group or salt thereof, or an organic group containing from 2 to 20 carbons, preferably 3 to 20 carbons, that is selected to form a 3-O-ester derivative; a 6-O-ester derivative; a 3-O,6-O-diester derivative; a 3-carbamate derivative; a 6-carbamate derivative; a 3,6-dicarbamate derivative; 3-carbonate derivative; a 6-carbamate derivative; or a 3,6-dicarbonate derivative. For example, in an embodiment, least one of R¹ and R² in formula (II) is selected from
PO₃H group or salt thereof;
n organic group containing from 2 to 20 carbons, preferably 3 to 20 carbons, that is selected to form a 3-O-ester derivative, a 6-O-ester derivative, or a 3-O,6-O-diester derivative; and
an organic group containing from 2 to 20 carbons that is selected to form a 3-carbomate derivative, a 6-carbamate derivative, a 3,6-dicarbamate derivative, a 3-carbonate derivative, a 6-carbonate derivative, or a 3,6-dicarbonate derivative.

Reference herein to compositions or methods that contain or utilize metalbolites of naltrexone will be understood to include such naltrexol prodrugs of the general formula (II).

Examples of 3-O-ester derivatives, 6-O-ester derivatives and 3-O, 6-O-diester derivatives of the formula (II) include mono- and di-methyl esters, mono- and di-ethyl esters, mono- and di-propyl esters, mono- and di-isopropyl esters, mono- and di-dimethylaminoethyl esters, mono- and di-tert-butyl esters, mono- and di-isobutyl esters, optionally arly-substituted mono- and di-phenyl esters, and mono- and di-succinyl esters. The phenyl esters may be optionally substituted on the aryl ring(s) with one or more substituents such as halo, C₁₋₆ alkoxy and C₁₋₆ alkyl. The esters may be mixed esters, e.g., 3-O-methyl, 6-O-ethyl ester of 6-β-naltrexol. In an embodiment, Formula (II) represents an ester or diester prodrug of a naltrexol (such as 6-β-naltrexol) in which one or neither of R¹ and R² is H and in which at least one of R¹ and R² is a group selected from the following:

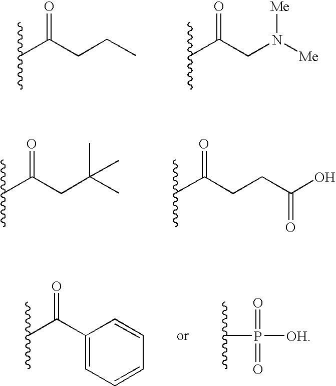

Examples of 3-carbamate derivatives, 6-carbamate derivatives and 3,6-dicarbamate derivatives of the formula (II) include mono- and di-methyl carbamates, mono- and di-ethyl carbamates, mono- and di-propyl carbamates, mono- and di-isopropyl carbamates, mono- and di-(dimethylamino)ethyl carbamates, mono- and di-tert-butyl carbamates, mono- and di-isobutyl carbamates, and optionally aryl-substituted mono- and di-phenyl carbamates. The phenyl carbamates may be optionally substituted on the aryl ring(s) with one or more substituents such as halo, C₁₋₆ alkoxy and C₁₋₆ alkyl. The carbamates may be mixed carbamates, e.g., 3-methyl, 6-ethyl carbamate of 6-β-naltrexol. In an embodiment, Formula (II) represents a carbamate or dicarbamate prodrug of naltrexol in which one or neither of R¹ and R² is H and in which at least one of R¹ and R² is a group selected from the following:

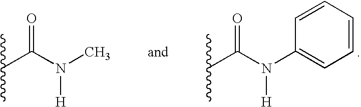

Examples of 3-carbonate derivatives, 6-carbonate derivatives and 3,6-dicarbonate derivatives of the formula (II) include mono- and di-methyl carbonates, mono- and di-ethyl carbonates, mono- and di-propyl carbonates, mono- and di-isopropyl carbonates, mono- and di-(dimethylamino)ethyl carbonates, mono- and di-tert-butyl carbonates, mono- and di-isobutyl carbonates, mono- and di-benzyl carbonates, and mono- and di-phenyl carbonates. The benzyl and phenyl carbonates may be optionally substituted on the aryl ring(s) with one or more substituents such as halo, C₁₋₆ alkoxy and C₁₋₆ alkyl. The carbonates may be mixed carbonates, e.g., 3-methyl, 6-ethyl carbonate of 6-β-naltrexol. In an embodiment, Formula (II) represents a carbonate or dicarbonate prodrug of naltrexol in which one or neither of R¹ and R² is H and in which at least one of R¹ and R² is a group selected from the following:

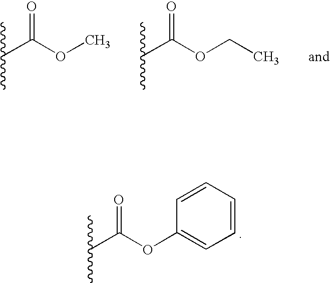

Prodrugs of the formula (II) can be made by reacting naltrexol with the appropriate derivatizing agent. Those skilled in the art, guided by the descriptions provided herein, will be able to make such prodrugs using routine experimentation. For example, 3-O-ester derivatives, 6-O-ester derivatives and 3-O, 6-O-diester derivatives of the formula (II) can be made in various ways. In an embodiment, the 3-O-ester derivatives can be prepared by dissolving 6-β-naltrexol in a suitable solvent (such as tetrahydrofuran (THF) or DMF), deprotonating by reacting with an equivalent of a base (e.g., solidum methoxide or lithium di-isopropyl amine) and reacting with an equivalent of the appropriate acylating agent, such as 2-dimethylaminoacetyl chloride or the appropriate acid anhydride (e.g., acetic, propionic, butyric, isobutyric, succinic, or benzoic anhydride) with stirring at room temperature or with gentle heating. Work up can be accomplished by partitioning between methylene chloride and aqueous sodium bicarbonate, following by drying over sodium sulfate and concentrating to yield the desired 3-O ester. The 6-O-ester derivatives can be prepared in a similar fashion. In an embodiment, the 3-O, 6-O-diester derivatives can be prepared by dissolving 6-β-naltrexol in a suitable solvent (such as methylene chloride or dimethylformamide (DMF)) and reacting it with an appropriate excess of 2-dimethylaminoacetyl chloride or the appropriate acid anhydride (e.g., 4 equivalents of acetic, propionic, butyric, isobutyric, succinic, or benzoic anhydride) with stirring at room temperature or with gentle heating in the presence of excess base (e.g., 4.5 equivalents of triethylamine). Work up can be accomplished by sodium bicarbonate extraction, following by drying over sodium sulfate and concentrating to yield the desired ester.

Dicarbamate prodrugs of the formula (II) can also be made in various ways. In an embodiment, the 3-carbamate derivatives can be prepared by dissolving 6-β-naltrexol in a suitable solvent (such as THF or DMF), deprotonating by reacting with a slight excess (e.g., 1.1 equivalent) of a base (e.g., lithium di-isopropyl amine) at 0° C., and reacting with a slight excess (e.g., 1.1 equivalent) of an appropriate isocyanate (e.g., methyl, ethyl, propyl, butyl or phenyl isocyanate) with stirring. Work up can be accomplished by partitioning between methylene chloride and aqueous sodium bicarbonate, following by drying over sodium sulfate and concentrating to yield the desired 3-carbamate. The corresponding 6-carbamate derivatives can be prepared in a similar fashion. In an embodiment, the 3-(dimethylamino)ethyl carbamates can be prepared by dissolving 6-β-naltrexol in a suitable solvent (such as THF or DMF), deprotonating by reacting with an equivalent of a base (e.g., lithium di-isopropyl amine) at 0° C., and reacting with a slight excess (e.g., 1.1 equivalent) of dimethylcarbamoyl chloride with stirring. Work up can be accomplished by partitioning between methylene chloride and aqueous sodium bicarbonate, following by drying over sodium sulfate and concentrating to yield the desired 3-(dimethylamino)ethyl carbamate. The corresponding 6-(dimethylamino)ethyl carbamate derivatives can be prepared in a similar fashion. In an embodiment, the 3, 6-dicarbamate derivatives can be prepared by dissolving 6-β-naltrexol in a suitable solvent (such as THF or DMF) and reacting with an excess of an appropriate isocyanate (e.g., 2.5 equivalents of methyl, ethyl, propyl, butyl or phenyl isocyanate) with stirring at room temperature or with gentle heating. Work up can be accomplished by partitioning between methylene chloride and aqueous sodium bicarbonate, following by drying the methylene chloride layer over sodium sulfate and concentrating to yield the desired 3, 6-dicarbamate. In an embodiment, the 3, 6-di-(dimethylamino)ethyl carbamates can be prepared by dissolving 6-β-naltrexol in a suitable solvent (such as THF or DMF) and reacting with an excess (e.g., 2.5 equivalents) of dimethylcarbamoyl chloride in the presence of excess base (e.g., 3 equivalents triethylamine) with stirring at 0° C. Work up can be accomplished by partitioning between methylene chloride and aqueous sodium bicarbonate, following by drying the methylene chloride layer over sodium sulfate and concentrating to yield the desired 3, 6-di-(dimethylamino)ethyl carbamate.

Dicarbonate prodrugs of the formula (II) can also be made in various ways. In an embodiment, the 3-carbonate derivatives can be prepared by dissolving 6-β-naltrexol in a suitable solvent (such as THF or DMF), deprotonating by treatment with a slight excess of a base (e.g., 1.2 equivalents of lithium di-isopropyl amine at 0° C.),then reacting with a slight excess of an appropriate chloroformate (e.g., 1.1 equivalents of methyl, ethyl, propyl, butyl or phenyl chloroformate). Work up can be accomplished by partitioning between methylene chloride and aqueous sodium bicarbonate, following by drying the methylene chloride layer over sodium sulfate and concentrating to yield the desired 3-carbonate. The corresponding 6-carbonate can be prepared in a similar fashion. In an embodiment, the 3, 6-dicarbonate derivatives can be prepared by dissolving 6-β-naltrexol in a suitable solvent (such as THF or DMF) and reacting with an excess of an appropriate chloroformate (e.g., 3 equivalents of methyl, ethyl, propyl, butyl or phenyl chloroformate) in the presence of excess base (e.g., 4 equivalents triethylamine) with stirring at 0° C. Work up can be accomplished by partitioning between methylene chloride and aqueous sodium bicarbonate, following by drying the methylene chloride layer over sodium sulfate and concentrating to yield the desired 3, 6-dicarbonate.

In some embodiments, the second compound in the pharmaceutical compositions described herein triggers the release of α-MSH. The second compound may increase the extracellular serotonin concentrations in the hypothalamus. In some embodiments, the second compound is selected from the group consisting of a selective serotonin reuptake inhibitor (SSRI), a serotonin 2C agonist, and a serotonin 1B agonist. In further embodiments, the second compound is selected, e.g., from the group consisting of fluoxetine, fluvoxamine, sertraline, paroxetine, citalopram, escitalopram, sibutramine, duloxetine, and venlafaxine, and pharmaceutically acceptable salts or prodrugs thereof.

The terms “serotonin 1B receptor,” “serotonin 2C receptor,” “5-HT1b receptor,” and “5-HT2c receptor” refer to receptors found more commonly in rodents. It is understood by those of skill in the art that other mammals have serotonin receptors on various neurons that are analogous in function and form to these receptors. Agonists or antagonists at these non-rodent, preferably human, serotonin receptors are within the scope of the present invention.

In certain embodiments, the second compound suppresses the expression of the AgRP gene or the production or release of agouti-related protein (AgRP). In some of these embodiments, the second compound suppresses the activity of neurons that express AgRP.

In other embodiments, the second compound suppresses the expression of the NPY gene or the production or release of neuropeptide Y (NPY). In some of these embodiments, the second compound suppresses the activity of neurons that express NPY. In further embodiments, the second compound is selected from the group consisting of NPY antagonists, ghrelin antagonists, and leptin. In certain other embodiments, the second compound agonizes the NPY Y2 receptor.

Other embodiments of the present invention include those in which the second compound is selected from the group consisting of a γ-amino butyric acid (GABA) inhibitor, a GABA receptor antagonist, and a GABA channel antagonist. By “GABA inhibitor” it is meant a compound that reduces the production of GABA in the cells, reduces the release of GABA from the cells, or reduces the activity of GABA on its receptors, either by preventing the binding of GABA to GABA receptors or by minimizing the effect of such binding. The GABA inhibitor may be a 5-HT1b agonist or another agent that inhibits the activity of NPY/AgRP/GABA neurons. In addition, the GABA inhibitor may suppress the expression of the AgRP gene, or the GABA inhibitor may suppress the production or release of AgRP. It is, however, understood that a 5-HT1b agonist may inhibit the NPY/AgRP/GABA neuron (and therefore activate POMC neurons) without acting as an inhibitor of the GABA pathway.

In certain other embodiments the GABA inhibitor increases the expression of the POMC gene. In some of these embodiments, the GABA inhibitor increases the production or release of pro-opiomelanocortin (POMC) protein. In certain other of these embodiments, the GABA inhibitor increases the activity on POMC expressing neurons. In some embodiments, the GABA inhibitor is topiramate.

In other embodiments the second compound is a dopamine reuptake inhibitor. Phretermine is an example of a dopamine reuptake inhibitor. In certain other embodiments, the second compound is a norepinephrine reuptake inhibitor. Examples of norepinephrine reuptake inhibitors include thionisoxetine and reboxetine. Other embodiments include those in which the second compound is a dopamine agonist. Some dopamine agonists that are commercially available include cabergoline, amantadine, lisuride, peroglide, ropinirole, pramipexole, and bromocriptine. In further embodiments, the second compound is a norepinephrine releaser, for example diethylpropion, or a mixed dopamine/norepinephrine reuptake inhibitor, for example, bupropion, and atomoxatine.

In certain other embodiments, the second compound is a 5-HT1b agonist, such as sumpatriptan, almotriptan, naratriptan, frovatriptan, rizatriptan, zomitriptan, and elitriptan.

In further embodiments, the second compound is an anticonvulsant. The anticonvulsant may be selected from the group consisting of zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide.

In certain embodiments, the second compound itself can be a combination of two or more compounds, or the second compound can be a single compound having two or more functions. For example, the second compound may be a combination of a dopamine reuptake inhibitor and a norepinephrine reuptake inhibitor, e.g. bupropion and mazindol. Alternatively, the second compound may be a combination of a SSRI and a norepinephrine reuptake inhibitor, such as sibutramine, venlafaxine, and duloxetine.

In certain embodiments, the second compound is an activator of the POMC neurons. Examples of POMC activators include Ptx1 and interleukin 1beta, (IL-1β).

In some embodiments, the first compound is 6-β-naltrexol or a prodrug of the formula (II) and the second compound is bupropion.

In another aspect, the present invention provides a method of affecting weight loss, comprising identifying an individual in need thereof and treating that individual to antagonize opioid receptor activity and to enhance α-MSH activity. In some embodiments, opioid receptor activity is antagonized by administering a metabolite of naltrexone or a pharmaceutically acceptable salt or prodrug thereof. In some embodiments, other metabolites of an opioid antagonist are used.

In some embodiments, the metabolite of naltrexone is 6-β-naltrexol.

In certain embodiments, the individual has a body mass index (BMI) greater than 25. In other embodiments, the individual has a BMI greater than 30. In still other embodiments, the individual has a BMI greater than 40. However, in some embodiments, the individual may have a BMI less than 25. In these embodiments, it may be beneficial for health or cosmetic purposes to affect weight loss, thereby reducing the BMI even further.

In some of the embodiments set forth above, α-MSH activity is enhanced by administering a second compound, where the compound triggers release of α-MSH or increases the activity of neurons that express α-MSH. The first compound is 6-β-naltrexol or a prodrug of the formula (II). In some embodiments, the second compound is a dopamine or norepinephrine reuptake inhibitor, which preferably is selected from group consisting of bupropion, thionisoxetine, atomoxetine, and reboxetine. In certain embodiments, the second compound is bupropion.

Individuals suffering from depression may gain weight as a result of their depression. In addition, certain depressed individuals gain weight as a side effect of the depression therapy. In certain embodiments, the method of invention set forth above is practiced with the proviso that the individual is not suffering from depression. In some embodiments, the individual's overweight state was not caused by treatment for depression.

Certain antidepressant drugs cause weight gain in individuals who take them. In some embodiments, the methods of the invention are directed to prevent weight gain associated with the administration of antidepressants. In other embodiments, the methods of the invention are directed to cause weight loss in individuals who have gained weight as the result of antidepressant therapy.

In some embodiments, the treating step of the above method comprises administering to the individual a first compound and a second compound, where the first compound is a metabolite of naltrexone, such as 6-β-naltrexol or a prodrug of the formula (II), and the second compound enhances α-MSH activity.

In some embodiments the first compound and the second compound are administered substantially simultaneously. In other embodiments the first compound is administered prior to the second compound. In yet other embodiments, the first compound is administered subsequent to the second compound. In other embodiments, one of the compounds is administered while the other compound is being administered.

In certain embodiments, the first compound and the second compound are administered individually. In other embodiments, the first compound and the second compound are covalently linked to each other such that they form a single chemical entity. The single chemical entity is then digested and is metabolized into two separate physiologically active chemical entities, one of which is the first compound and the other one is the second compound.

In some embodiments, the compositions of the present invention are selected from the following combinations of compounds:
a SSRI in combination with 6-β-naltrexol;
serotonin in combination with 6-β-naltrexol;
a dopamine reuptake inhibitor in combination with 6-β-naltrexol;
a dopamine/norepinephrine reuptake inhibitor in combination with 6-β-naltrexol;
a dopamine agonist in combination with 6-β-naltrexol;
a compound of the formula (II) in combination with bupropion.

Examples of norepinephrine agonists include phendimetrazine and benzphetamine. Examples of adenosine compounds include all xanthine derivatives, such as adenosine, theophylline, theobromine, and aminophylline. An example of a cholinergic receptor antagonist is nicotine.

In another aspect, the present invention provides a method of increasing satiety in an individual comprising identifying an individual in need thereof and treating that individual to antagonize opioid receptor activity with a metabolite of naltrexone and to ehance α-MSH activity.

In some embodiments, treating the individual comprises administering to the individual a first compound and a second compound, where the first compound is a metabolite of naltrexone and the second compound enhances α-MSH activity.

In some embodiments the first compound and the second compound are administered substantially simultaneously. In other embodiments the first compound is administered prior to the second compound. In yet other embodiments, the first compound is administered subsequent to the second compound.

In yet another aspect, the present invention provides a method of suppressing the appetite of an individual comprising identifying an individual in need thereof and treating that individual to antagonize opioid receptor activity and to enhance α-MSH activity. In some embodiments, treating the individual comprises administering to the individual a first compound and a second compound, where the first compound is a metabolite of naltrexone and the second compound enhances α-MSH activity.

In some embodiments, the metabolite of naltrexone is 6-β-naltrexol.

In another aspect, the present invention provides a method of increasing energy expenditure in an individual comprising identifying an individual in need thereof and treating that individual to antagonize opioid receptor activity and to enhance α-MSH activity. In some embodiments, treating the individual comprises administering to the individual a first compound and a second compound, where the first compound is a metabolite of naltrexone and the second compound enhances α-MSH activity.

In certain embodiments disclosed herein, an individual is given a pharmaceutical composition comprising a combination of two or more compounds to affect weight loss. In some of these embodiments, each compound is a separate chemical entity. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

Thus, in another aspect, the present invention provides synthetic routes to novel molecules in which 6-β-naltrexol is linked by a flexible linker to a selective serotonin reuptake inhibitor (SSRI).

In another aspect, the invention relates to a pharmaceutical composition comprising a combination of a metabolite of naltrexone and a compound that causes increased agonism of MC3-R and/or MC4-R compared to normal physiological conditions, as described above, or comprising a linked molecule, as described herein, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by forming derivatives of the compounds described herein, e.g., by reacting with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” refers to chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa, 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions as described herein may be formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane; carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions.

Alternatively, the active ingredient may be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a an effective or therapeutically effective amount means an amount of compound effective to prevent, alleviate or ameliorate symptoms of disease or prolong the survival of the subject being treated. Determination of an effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. See e.g., Fingl et al. 1975, in “The Pharmacological Basis of Therapeutics”, Ch. 1 p. 1. Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

In some embodiments, the amount of the naltrexone metabolite (e.g., a natrexol such as 6-β-naltrexol, 2-hydroxy-3-methoxy-6-b-naltrexol, or 2-hydroxy-3-methyl-naltrexone, or a naltrexol prodrug) is minimized, but is present in an amount sufficient to allow for a beneficial effect on weight or on the appetite of the subject. In some embodiments, the amount of the naltrexone metabolite is an amount effective to reduce weight, and that amount is less than the amount of naltrexone or other non-6-β-naltrexol opioid antagonist that might otherwise be used. In some embodiments, the amount of the naltrexone metabolite used is an amount no more than the amount that induces unwanted side effects. In some embodiments, the naltrexone metabolite is administered in an amount that does not induce side-effects associated with naltrexone, for example, side effects that occur at doses of 50-100 mg of naltrexone. In some embodiments, the side-effects include nausea, headache, dizziness, fatigue, insomnia, anxiety, and sleepiness.

In some embodiments, the amount of naltrexone metabolite administered or in the composition is between 800 and 50 mg. In other embodiments, the amount of naltrexone metabolite is between 1 and 50 mg, for example 1-5, 5-10, 10-15, 15-20, 20-25, 25-30, 30-35, 35-40, 40-45, and 45-50 mg. In some embodiments, the amount of naltrexone metabolite used is more than an amount of naltrexone that can be used without detrimental side-effects, for example, more than 50 mg of 6-β-naltrexol can be used. For example, in some embodiments, the amount of naltrexone metabolite used is 50 mg, 50-100 , 100-150, 150-200, 200-250, 250-300, 300-350, 350-400, or more milligrams of 6-β-naltrexol. In some embodiments, the fact that a naltrexone metabolite (instead of naltrexone) is utilized is advantageous in the sense that the method or means of administration of the first compound to the patient may be adjusted. For example, a method that might normally take longer for the compound to be effective, e.g., patch delivery (e.g., compared to I.V.) can be used to administer the naltrexone metabolite in a situation where a faster method might be required using naltrexone itself.

Dosage amount and interval may be adjusted individually to provide plasma levels of the active moiety that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention.

### SOME EMBODIMENTS OF THE INVENTION

Some of the embodiments of the present invention are as follows:

In the first embodiment, the invention relates to a composition for affecting weight loss comprising a first compound and a second compound, wherein said first compound is a naltrexone metabolite and said second compound causes 1) increased agonism of MC3-R and/or MC4-R compared to normal physiological conditions, and/or 2) enhances alpha-MSH activity. In a preferred embodiment of the first embodiment, the first compound is 6-β-naltrexol.

In the second embodiment, the invention relates to the composition of the first embodiment, wherein the first compound antagonizes an opioid receptor in a mammal.

In the third embodiment, the invention relates to the composition of the second embodiment, wherein said opioid receptor is selected from a μ-opioid receptor (MOP-R), a κ-opioid receptor, and a δ-opioid receptor.

In the fourth embodiment, the invention relates to the composition of the second embodiment, wherein said first compound antagonizes a μ-opioid receptor (MOP-R) in a mammal.

In the fifth embodiment, the invention relates to the composition of the first embodiment, wherein said second compound triggers the release of α-melanocyte stimulating hormone (α-MSH).

In the sixth embodiment, the invention relates to the composition of the fifth embodiment, wherein said second compound increases the extracellular serotonin concentrations in the hypothalamus.

In the seventh embodiment, the invention relates to the composition of the sixth embodiment, wherein said second compound is selected from the group consisting of a selective serotonin reuptake inhibitor (SSRI), a serotonin 2C agonist, and a serotonin 1B agonist.

In the eighth embodiment, the invention relates to the composition of the seventh embodiment, wherein said second compound is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

In the ninth embodiment, the invention relates to the composition of the first embodiment, wherein said second compound suppresses the expression of the AgRP gene or the production or release of agouti-related protein (AgRP).

In the tenth embodiment, the invention relates to the composition of the first embodiment, wherein said second compound suppresses the activity of neurons that express AgRP.

In the eleventh embodiment, the invention relates to the composition of the first embodiment, wherein said second compound suppresses the expression of the NPY gene or the production or release of neuropeptide Y (NPY).

In the twelfth embodiment, the invention relates to the composition of the first embodiment, wherein said second compound suppresses the activity of neurons that express NPY.

In the thirteenth embodiment, the invention relates to the composition of the first embodiment, wherein said second compound is selected from the group consisting of NPY Y1 receptor antagonists, ghrelin antagonists, and leptin.

In the fourteenth embodiment, the invention relates to the composition of the first embodiment, wherein said second compound agonizes NPY Y2 receptor.

In the fifteenth embodiment, the invention relates to the composition of the first embodiment, wherein said second compound is selected from the group consisting of a γ-amino butyric acid (GABA) inhibitor, a GABA receptor antagonist, and a GABA channel antagonist.

In the sixteenth embodiment, the invention relates to the composition of the fifteenth embodiment, wherein said GABA inhibitor is a 5-HT1b agonist, which may be selection from sumatriptan, almotriptan, naratriptan, frovatriptan, rizatriptan, zomitriptan, and elitriptan.

In the seventeenth embodiment, the invention relates to the composition of the fifteenth embodiment, wherein said GABA inhibitor suppresses the expression of the AgRP gene.

In the eighteenth embodiment, the invention relates to the composition of the fifteenth embodiment, wherein said GABA inhibitor suppresses the production or release AgRP gene.

In the nineteenth embodiment, the invention relates to the composition of the fifteenth embodiment, wherein said GABA inhibitor increases the expression of the POMC gene.

In the twentieth embodiment, the invention relates to the composition of the fifteenth embodiment, wherein said GABA inhibitor increases the production or release of α-MSH from pro-opiomelanocortin (POMC) neurons.

In the twenty first embodiment, the invention relates to the composition of the fifteenth embodiment, wherein said GABA inhibitor increases the activity of POMC expressing neurons. In a preferred embodiment of the twenty-first embodiment, the GABA inhibitor is topiramate.

In the twenty second embodiment, the invention relates to the composition of the first embodiment, wherein said second compound is a dopamine reuptake inhibitor.

In the twenty third embodiment, the invention relates to the composition of the twenty second embodiment, wherein said dopamine reuptake inhibitor is phentermine.

In the twenty fourth embodiment, the invention relates to the composition of the first embodiment, wherein said second compound is a norepinephrine reuptake inhibitor.

In the twenty fifth embodiment, the invention relates to the composition of the twenty fourth embodiment, wherein said norepinephrine reuptake inhibitor is selected from bupropion, thionisoxetine, and reboxetine.

In the twenty sixth embodiment, the invention relates to the composition of the first embodiment, wherein said second compound is a dopamine agonist.

In the twenty seventh embodiment, the invention relates to the composition of the twenty sixth embodiment, wherein said dopamine agonist is selected from the group consisting of cabergoline, amantadine, lisuride, pergolide, ropinirole, pramipexole, and bromocriptine.

In the twenty eighth embodiment, the invention relates to the composition of the first embodiment, wherein said second compound is a norepinephrine releaser.

In the twenty ninth embodiment, the invention relates to the composition of the twenty eighth embodiment, wherein said norepinephrine releaser is diethylpropion.

In the thirtieth embodiment, the invention relates to the composition of the first embodiment, wherein said second compound is a combination of a dopamine reuptake inhibitor and a norepinephrine reuptake inhibitor.

In the thirty first embodiment, the invention relates to the composition of the thirtieth embodiment, wherein said second compound is selected from bupropion and mazindol.

In the thirty second embodiment, the invention relates to the composition of the first embodiment, wherein said second compound is a combination of a SSRI and a norepinephrine reuptake inhibitor.

In the thirty third embodiment, the invention relates to the composition of the thirty second embodiment, wherein said second compound is selected from sibutramine, venlafaxine, and duloxetine.

In the thirty fourth embodiment, the invention relates to the composition of the first embodiment, wherein said second compound is fluoxetine.

In the thirty fifth embodiment, the invention relates to the composition of the thirty fourth embodiment, wherein the naltrexone metabolite (e.g., 6-β-naltrexol or a compound of the formula (II)) is in a time-release formulation whereas the fluoxetine is in an immediate release formulation.

In the thirty sixth embodiment, the invention relates to a method of affecting weight loss, comprising identifying an individual in need thereof and treating that individual to antagonize opioid receptor activity through the administration of a naltrexone metabolite and the enhancement of α-MSH activity. In a preferred embodiment of the thirty sixth embodiment, the naltrexone metabolite is 6-β-naltrexol or a compound of the formula (II).

In the thirty seventh embodiment, the invention relates to the method of the thirty sixth embodiment, wherein said individual has a body mass index greater than 25.

In the thirty eighth embodiment, the invention relates to the method of the thirty sixth embodiment, further comprising administering a partial opioid agonist selected from the group consisting of pentacozine, buprenorphine, nalorphine, propiram, and lofexidine.

In the thirty ninth embodiment, the invention relates to the method of the thirty sixth embodiment through the thirty eighth embodiment, wherein α-MSH activity is enhanced by administering a compound, wherein said compound triggers release of α-MSH or increase the activity of neurons that express α-MSH.

In the fortieth embodiment, the invention relates to the method of the thirty ninth embodiment, wherein said compound is a selective serotonin reuptake inhibitor (SSRI) or a specific 5-HT receptor agonist.

In the forty first embodiment, the invention relates to the method of the fortieth embodiment, wherein said 5-HT receptor is selected from 5-HT1b receptor and 5-HT2c receptor.

In the forty second embodiment, the invention relates to the method of the fortieth embodiment, wherein said SSRI is selected from fluoxetine, fluvoxamine, sertraline, paroxetine, citalopram, escitalopram, sibutramine, duloxetine, and venlafaxine, and pharmaceutically acceptable salts or prodrugs thereof.

In the forty third embodiment, the invention relates to the method of the thirty nine embodiment, wherein said second compound is a y-amino butyric acid (GABA) inhibitor.

In the forty fourth second embodiment, the invention relates to the method of the forty third embodiment, wherein said GABA inhibitor is a 5-HT1b receptor agonist.

In the forty fifth embodiment, the invention relates to the method of the forty third embodiment, wherein said GABA inhibitor suppresses the expression of the AgRP gene.

In the forty sixth embodiment, the invention relates to the method of the forty third embodiment, wherein said GABA inhibitor suppresses the production or release of AgRP.

In the forty seventh embodiment, the invention relates to the method of the forty third embodiment, wherein said 5-HT agonists inhibits the NPY/AgRP/GABA neurons.

In the forty eighth embodiment, the invention relates to the method of the forty third embodiment, wherein said GABA inhibitor suppresses the activity of neurons that express AgRP.

In the forty ninth embodiment, the invention relates to the method of the forty third embodiment, wherein said GABA inhibitor is topiramate.

In the fiftieth embodiment, the invention relates to the method of the thirty ninth embodiment, wherein said compound is selected from the group consisting of a dopamine reuptake inhibitor, a norepinephrine reuptake inhibitor, a dopamine agonist, a norepinephrine releaser, a combination of a dopamine reuptake inhibitor and a norepinephrine reuptake inhibitor, and a combination of a SSRI and a norepinephrine reuptake inhibitor.

In the fifty first embodiment, the invention relates to the method of the fiftieth embodiment, wherein said compound is not phentermine.

In the fifty second embodiment, the invention relates to the method of the thirty sixth embodiment, with the proviso that the individual is not suffering from Prader-Willi syndrome.

In the fifty third embodiment, the invention relates to the method of the thirty sixth embodiment, with the proviso that α-MSH is not stimulated with fluoxetine.

In the fifty fourth embodiment, the invention relates to the method of the thirty sixth embodiment, wherein said treating step comprises administering to said individual a first compound and a second compound, wherein said first compound is an opioid antagonist and said second compound enhances α-MSH activity.

In the fifty fifth embodiment, the invention relates to the method of the fifty fourth embodiment, wherein said first compound and said second compound are administered nearly simultaneously.

In the fifty sixth embodiment, the invention relates to the method of the fifty fifth embodiment, wherein said first compound is administered prior to said second compound.

In the fifty seventh embodiment, the invention relates to the method of the fifty fifth embodiment, wherein said first compound is administered subsequent to said second compound.

In the fifty eighth embodiment, the invention relates to a method of increasing satiety in an individual comprising identifying an individual in need thereof and treating that individual with a first compound, said first compound comprising a naltrexone metabolite to antagonize opioid receptor activity and a second compound to enhance α-MSH activity. In a preferred embodiment of the fifty eighth embodiment, said naltrexone metabolite is 6-β-naltrexol. In another preferred embodiment of the fifty eighth embodiment, said naltrexone metabolite is a compound of the formula (II).

In the fifty ninth embodiment, the invention relates to the method of the fifty eighth embodiment, wherein said first compound and said second compound are administered substantially simultaneously.

In the sixtieth embodiment, the invention relates to the method of the fifty eighth embodiment, wherein said first compound is administered prior to said second compound.

In the sixty first embodiment, the invention relates to the method of the fifty eighth embodiment, wherein said first compound is administered subsequent to said second compound.

In the sixty second embodiment, the invention relates to a method of increasing energy expenditure in an individual comprising identifying an individual in need thereof and treating that individual to antagonize opioid receptor activity and to enhance α-MSH activity, wherein treating the individual comprises administering to said individual a first compound and a second compound, wherein said first compound is a naltrexone metabolite and said second compound enhances α-MSH activity. In a preferred embodiment of the sixty second embodiment, the naltrexone metabolite is 6-β-naltrexol. In another preferred embodiment of the sixty second embodiment, the naltrexone metabolite is a compound of the formula (II).

In the sixty third embodiment, the invention relates to the method of the sixty second embodiment, wherein said first compound and said second compound are administered nearly simultaneously.

In the sixty fourth embodiment, the invention relates to the method of the sixty second embodiment, wherein said first compound is administered prior to said second compound.

In the sixty fifth embodiment, the invention relates to the method of the sixty second embodiment, wherein said first compound is administered subsequent to said second compound.

In the sixty sixth embodiment, the invention relates to a method of suppressing the appetite of an individual comprising identifying an individual in need thereof and treating that individual to antagonize opioid receptor activity and to enhance α-MSH activity, wherein treating the individual comprises administering to said individual a first compound and a second compound, wherein said first compound is a naltrexone metabolite and said second compound enhances α-MSH activity. In a preferred embodiment of the sixty sixth embodiment, the naltrexone metabolite is 6-β-naltrexol. In another preferred embodiment of the sixty sixth embodiment, the naltrexone metabolite is a compound of the formula (II).

In the sixty seventh embodiment, the invention relates to the method of the sixty sixth embodiment, wherein said first compound and said second compound are administered nearly simultaneously.

In the sixty eighth embodiment, the invention relates to the method of the sixty sixth embodiment, wherein said first compound is administered prior to said second compound.

In the sixty ninth embodiment, the invention relates to the method of the sixty sixth embodiment, wherein said first compound is administered subsequent to said second compound.

In the seventieth embodiment, the invention relates to a method of affecting weight loss in an individual comprising identifying an individual in need thereof and treating that individual with a combination of a naltrexone metabolite and an SSRI, provided that the individual does not suffer from Prader-Willi syndrome or binge eating disorder. In a preferred embodiment of the seventieth embodiment, the naltrexone metabolite is 6-β-naltrexol or a compound of the formula (II) and the SSRI is fluoxetine.

In the seventy first embodiment, the invention relates to the method of the seventieth embodiment, wherein the individual has a BMI greater than 30.

In the seventy second embodiment, the invention relates to the method of the seventieth embodiment, wherein the individual has a BMI greater than 25.

In the seventy third embodiment, the invention relates to the method of the seventieth embodiment, wherein the naltrexone metabolite (e.g., 6-β-naltrexol or a compound of the formula (II)) is in a time-release formulation whereas the SSRI (e.g., fluoxetine) is in an immediate release formulation.

In the seventy fourth embodiment, the invention relates to the method of the seventy third embodiment, wherein the plasma concentration level of both the naltrexone metabolite and SSRI follow a similar concentration profile.

In the seventy fifth embodiment, the invention relates to the method of the seventy third embodiment, wherein the naltrexone metabolite and the SSRI are administered substantially simultaneously.

In the seventy sixth embodiment, the invention relates to the method of the seventy third embodiment, wherein the naltrexone metabolite is administered prior to the SSRI.

In the seventy seventh embodiment, the invention relates to the method of the seventy third embodiment, wherein the naltrexone metabolite is administered subsequent to the SSRI.

In the seventy eighth embodiment, the invention relates to any of the above methods, wherein the amount of naltrexone metabolite administered is less than an amount of naltrexone that would have to be administered to achieve substantially the same amount of weight loss.

In the seventy ninth embodiment, the invention relates to any of the above methods, wherein the amount of naltrexone metabolite (e.g., 6-β-naltrexol or compound of the formula (II)) administered is selected from the group consisting of 50, 45, 40, 35, 30, 25, 20, 15, 10, 5, and 1 mg.

In the eightieth embodiment, the invention relates to any of the above compositions, wherein the amount of naltrexone metabolite (e.g., 6-β-naltrexol or compound of the formula (II)) in the composition is less than an amount of naltrexone that would have to be administered to achieve substantially the same amount of weight loss with the particular composition.

In the eighty first embodiment, the invention relates to any of the above compositions, wherein the amount of naltrexone metabolite (e.g., 6-β-naltrexol or compound of the formula (II)) in the composition is selected from the group consisting of 50, 45, 40, 35, 30, 25, 20, 15, 10, 5, and 1 mg.

In the eighty second embodiment, the invention relates to a method of affecting weight loss comprising administering to the patient or subject in need of treatment an effective amount of a metabolite of naltrexone. In a preferred embodiment, the naltrexone metabolite is 6-β-naltrexol.

In the eighty third embodiment, the invention relates to a method or composition of any one of the first through eighty second embodiments in which the naltrexone metabolite is a prodrug, e.g., a compound of the formula (II).

In the eighty fourth embodiment, the invention relates to a method or composition of any one of the first through eighty third embodiments in which the 6-β-naltrexol is a prodrug.

In the eighty fifth embodiment, the invention relates to a compound of the formula (II) in which at least one of R¹ and R² is selected from a PO₃H group or slat thereof; an organic group containing from 2 to 20 carbons, preferably 3 to 20 carbons, that is selected to form a 3-O-ester derivative, a 6-O-ester derivative, or a 3-O,6-O-diester derivative; and an organic group containing from 2 to 20 carbons, preferably 3 to 20 carbons, that is selected to form a 3- carbamate derivative, a 6- carbamate derivative, a 3,6-dicarbamate derivative, 3-carbonate derivative, a 6-carbonate derivative or a 3,6-dicarbonate derivative.

In the eighty sixth embodiment, the invention relates to a compound of the eighty fifth embodiment in which one or neither of R¹ and R² is H and in which at least one of R¹ and R² is a group selected from the following:

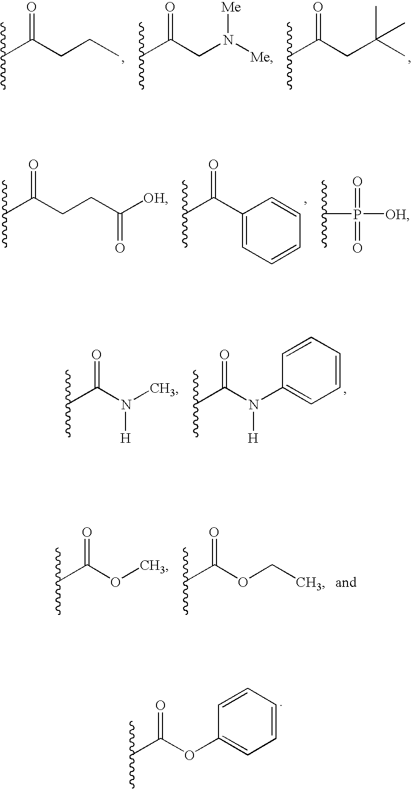

In the eighty seventh embodiment, the invention relates to a method or composition of any one of the above embodiments, wherein said naltrexone metabolite is the compound of the eighty fifth or eighty sixth embodiment.

In an eighty eighth embodiment, the invention relates to a method of manufacturing a medicament using any of the compounds or compositions disclosed herein for the treatment of a disease, condition, or for affecting a beneficial outcome where the disease, condition of beneficial outcome includes: obesity, affecting weight loss, increasing energy expenditure, increasing satiety in an individual, or suppressing the appetite of an individual.

### EXAMPLES

The examples below are non-limiting and are merely representative of various aspects of the invention.

### Example 1

Combination of 6-β-naltrexol and Bupropion:

Individuals having a BMI of greater than 25 are identified. Each individual is instructed to take 20 to 50 mg of 6-β-naltrexol on a daily basis. In addition, each individual is instructed to take bupropion. The usual adult dose is 300 mg per day, given three times daily. Dosing should begin at 200 mg per day, given as 100 mg twice daily. Based on clinical response, this dose may be increased to 300 mg per day, given as 100 mg three times daily. No single dose is to exceed 150 mg.

The individuals are monitored for a period of months. It is recommended that the dosage be adjusted so that each individual loses weight at a rate of 10% of initial weight every 6 months. However, the rate of weigh loss for each individual may be adjusted by the treating physician based on the individual's particular needs.

### Example 2

Combinations with 6-β-naltrexol:

In a multicenter, randomized, blinded, placebo-controlled clinical trial with 6 groups, the following drug combinations are tested:
Group 1: Fluoxetine 60 mg po QD plus 6-β-naltrexol 50 mg po QD
Group 2: Fluoxetine 60 mg po QD plus N-placebo po QD
Group 3: Bupropion-SR 150 mg po BID plus 6-β-naltrexol 50 mg po QD
Group 4: Bupropion-SR 150 mg po BID plus N-placebo po QD
Group 5: P-placebo po BID plus 6-β-naltrexol 50 mg po QD
Group 6: P-placebo po BID plus N-placebo po QD

In any of the above groups, the dosage of fluoxetine may be in the range between 6 mg and 60 mg, for example, 6 mg, 10 mg, 12 mg, 18 mg, 20 mg, 24 mg, 30 mg, 36 mg, 40 mg, 42 mg, 45 mg, 48 mg, 54 mg, and 60 mg. Bupropion may be administered in doses in the range between 30 mg and 300 mg, for example, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg. 6-β-naltrexol may be administered in doses in the range between 5 mg and 50 mg, for example, 5 mg, 10 mg, 15 mg, 20 mg, 25 mg, 30 mg, 35 mg, 40 mg, 45 mg, and 50 mg.

Subjects are evaluated as out-patients during this study. All subjects in this trial receive diet instruction, behavior modification advice and instruction to increase their activity, a regimen shown to give weight loss. Subjects are randomized to receive study drugs in various combinations.

Subjects in groups 5 and 6 cross-over to treatment with fluoxetine plus 6-β-naltrexol or bupropion SR plus 6-β-naltrexol after week 16 for the extension treatment period which provide additional data on safety of the combination therapies.

The primary endpoint is percent and absolute change from baseline in body weight at 16 weeks. Secondary endpoints include weight loss at 24, 36, and 48 weeks, number and proportion of subjects who achieve at least a 5% weight loss and a 10% weight loss (responder analysis), changes in obesity-associated cardiovascular risk factors (total cholesterol, LDL cholesterol, HDL cholesterol, triglycerides, glucose and insulin) and waist circumference, and safety and tolerability. Adverse events, laboratory parameters, vital signs, and the Hospital Anxiety and Depression (HAD) Scale are used to monitor safety and tolerability.

### Example 3

6-β-naltrexol vs. Naltrexone in Weight Loss:

Individuals having a BMI of greater than 25 are identified. The individuals are divided into two groups. The first group is instructed to take one 50 mg tablet of 6-β-naltrexol on a daily basis. The second group is instructed to take one 50 mg tablet of naltrexone on a daily basis.

The individuals are monitored for a period of months. It is recommended that the dosage be adjusted so that each individual loses weight at a rate of 10% of initial weight every 6 months. However, the rate of weight loss for each individual may be adjusted by the treating physician based on the individual's particular needs.

If the initial dosage is not effective, then either dosage can be increased by 20 mg per day, though never exceeding 80 mg total per day. If the initial dosage results in a more rapid weight loss than the above rate, the dosage of either can be reduced.

This can demonstrate that 6-β-naltrexol can be more effective in promoting weight loss than naltrexone.

### Example 4

Dose of Allowable 6-β-naltrexol:

This example will allow one to determine the range of safe levels of 6-β-naltrexol to administer to a patient. A patient is first administered 40 mg of naltrexol and then examined over a period of time to determine if there are any unwanted side-effects. Following this time period, if there are no unwanted side effects, the dosage is increased to 50, 60, 70 mg, etc. to determine the amount of 6-β-naltrexol that a patient can receive. In addition to the examination for unwanted side effects (e.g., those displayed from high doses of naltrexone) the patient's weight can also be examined. In particular, any decrease in weight of the patient can be monitored as well as any decrease in patient appetite. This can further allow for the effectiveness of 6-β-naltrexol to be examined in the patient.

Following these initial rounds, a second compound, e.g., one that enhances alpha-MSH activity, can be added and similarly titrated, with any unwanted side effects and loss in weight or appetite of the subject being examined.

In the alternative, instead of raising the level of 6-β-naltrexol, one can titrate the amount downward and thereby obtain a minimal amount of 6-β-naltrexol required to obtain the favorable results.

It will be appreciated by those skilled in the art that various modifications and changes can be made without departing from the scope of the embodiments disclosed herein. Such modifications and changes are intended to fall within the scope of the embodiments disclosed herein, as defined by the appended claims.

What is claim is:

1. A composition for affecting weight loss comprising a first compound and a second compound;
wherein said first compound is a metabolite of naltrexone; and
wherein said second compound increases agonism of a melanocortin 3 receptor (MC3-R) or melanocortin 4 receptor (MC4-R), or increases the concentration of α-MSH in the central nervous system.

2. The composition of claim 1, wherein said second compound is bupropion.

3. The composition of claim 1, wherein said first compound is 6-β-naltrexol.

4. The composition of claim 1, wherein said first compound is a prodrug of the naltrexone metabolite.

5. The composition of claim 4, wherein said first compound is a compound of the formula (II): 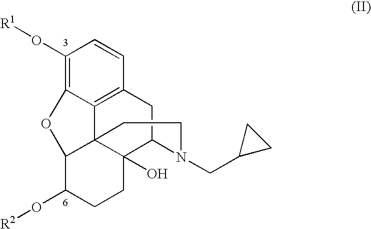
wherein at least one of R¹ and R² is a PO₃H group or a salt thereof, or an organic group containing from 2 to 20 carbons that is selected to form a 3-O-ester derivative, a 6-O-ester derivative, a 3-O,6-O-diester derivative, a 3-carbamate derivative, a 6-carbamate derivative, a 3,6-dicarbamate derivative, a 3-carbonate derivative, a 6-carbonate derivative, or a 3,6-dicarbonate derivative.

6. The composition of claim 5, wherein one or neither of R¹ and R² is H; and
wherein at least one of R¹ and R² is a group selected from the following:

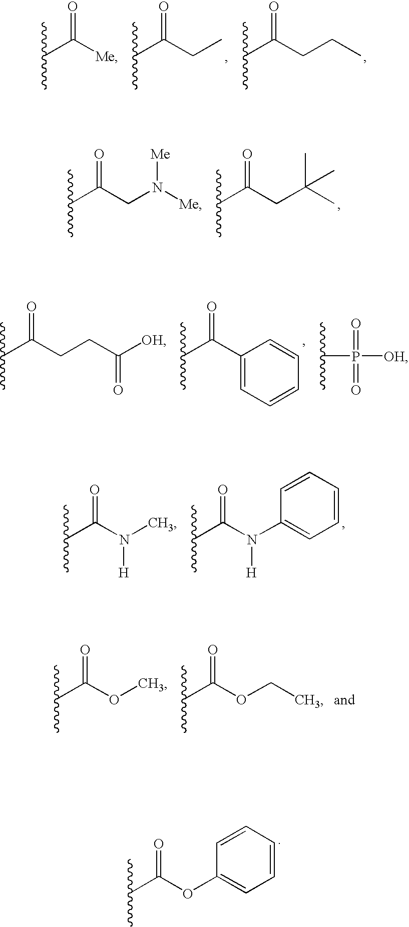

7. The composition of claim 1, wherein said second compound is selected from the group consisting of a selective serotonin reuptake inhibitor (SSRI), a serotonin 2C agonist, a serotonin 1B agonist, a γ-amino butyric acid (GABA) inhibitor, a GABA receptor antagonist, a GABA channel antagonist, an anticonvulsant, a dopamine agonist, a dopamine reuptake inhibitor, a norepinephrine reuptake inhibitor, a norepinephrine releaser, and a norepinephrine agonist.

8. The composition of claim 7, wherein said second compound is selected from the group consisting of fluoxetine, fluvoxamine, sertraline, paroxetine, citalopram, escitalopram, sibutramine, duloxetine, venlafaxine, sumatriptan, almotriptan, naratriptan, frovatriptan, rizatriptan, zomitriptan, elitriptan, zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, ethosuxmide, cabergoline, amantadine, lisuride, pergolide, ropinirole, pramipexole, bromocriptine, phentermine, bupropion, thionisoxetine, reboxetine, diethylpropion, phendimetrazine, benzphetamine, and pharmaceutically acceptable salts or prodrugs thereof.

9. The composition of claim 5, wherein said second compound is bupropion.

10. A method of affecting weight loss, comprising:
identifying an individual in need thereof;
administering an effective amount of the composition of claim 1.

11. The method of claim 10, wherein the first compound is 6-β-naltrexol or a prodrug of the naltrexone metabolite.

12. The method of claim 11, wherein the second compound is bupropion.

13. A compound of the formula (II): 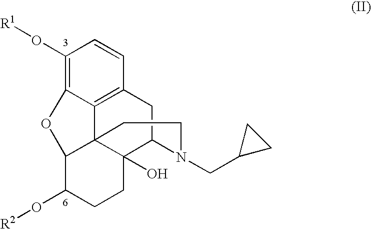
wherein one or neither of R¹ and R² is H; and
wherein at least one of R¹ and R² is a group selected from the following:

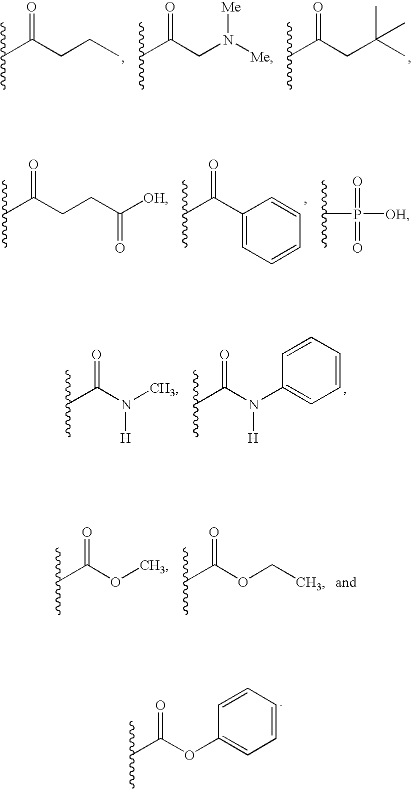

14. A method of affecting weight loss, comprising:
identifying an individual in need thereof;
administering an effective amount of the compound of claim 13.

15. The method of claim 14, further comprising administering an effective amount of a second compound that increases agonism of a melanocortin 3 receptor (MC3-R) or melanocortin 4 receptor (MC4-R), or increases the concentration of α-MSH in the central nervous system.

16. The method of claim 15, wherein the second compound is bupropion.

17. A composition for affecting weight loss comprising an effective amount of a compound of claim 13 and a pharmaceutically acceptable carrier.

18. The composition of claim 17, further comprising an effective amount of a second compound that increases agonism of a melanocortin 3 receptor (MC3-R) or melanocortin 4 receptor (MC4-R), or increases the concentration of α-MSH in the central nervous system.

19. The composition of claim 18, wherein the second compound is bupropion.

20. A method of affecting weight loss, comprising:
identifying an individual in need thereof;
administering an effective amount of the composition of claim 18.

21. A method of affecting weight loss, comprising:
identifying an individual in need thereof; and
administering an effective amount of a first compound and a second compound;
wherein said first compound is a metabolite of naltrexone and wherein said second compound increases agonism of a melanocortin 3 receptor (MC3-R) or melanocortin 4 receptor (MC4-R), or increases the concentration of α-MSH in the central nervous system.

22. The method of claim 21, wherein the first compound and the second compound are administered substantially simultaneously.

23. The method of claim 21, wherein the first compound is administered prior to the second compound.

24. The method of claim 21, wherein the first compound is administered subsequent to the second compound.

25. The method of claim 21, wherein said first compound is 6-β-naltrexol.

26. The method of claim 21, wherein said first compound is a prodrug of a naltrexone metabolite.

27. The method of claim 26, wherein said first compound is a compound of the formula (II): 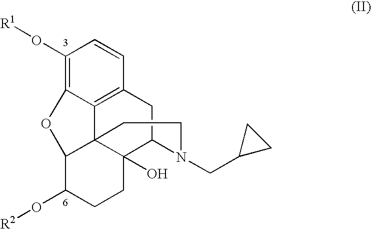
wherein at least one of R¹ and R² is a PO₃H group or salt thereof, or an organic group containing from 2 to 20 carbons that is selected to form a 3-O-ester derivative, a 6-O-ester derivative, a 3-O,6-O-diester derivative, a 3-carbamate derivative, a 6-carbamate derivative, a 3,6-dicarbamate derivative, a 3-carbonate derivative, a 6-carbonate derivative, or a 3,6-dicarbonate derivative.

28. The method of claim 21, wherein said second compound is selected from the group consisting of a selective serotonin reuptake inhibitor (SSRI), a serotonin 2C agonist, and a serotonin 1B agonist.

29. The method of claim 28, wherein said second compound is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

30. The method of claim 28, wherein said second compound is selected from the group consisting of sumatriptan, almotriptan, naratriptan, frovatriptan, rizatriptan, zomitriptan, and elitriptan.

31. The method of claim 21, wherein said second compound is selected from the group consisting of a γ-amino butyric acid (GABA) inhibitor, a GABA receptor antagonist, a GABA channel antagonist and an anticonvulsant.

32. The method of claim 31, wherein said second compound is selected from the group consisting of zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide.

33. The method of claim 21, wherein said second compound is selected from the group consisting of a dopamine agonist, a dopamine reuptake inhibitor, a norepinephrine reuptake inhibitor, a norepinephrine releaser, and a norepinephrine agonist.

34. The method of claim 33, wherein said second compound is selected from the group consisting of cabergoline, amantadine, lisuride, pergolide, ropinirole, pramipexole, bromocriptine, phentermine, bupropion, thionisoxetine, reboxetine, diethylpropion, phendimetrazine and benzphetamine.

35. The method of claim 34, wherein said second compound is bupropion.
